SEQUENCE LISTING (7 sequences)

SEQ ID NO: 1 - protein, 44 aa, Artificial sequence
  GRF peptide
XXDAIFTXSYRKXLXQLXARKLLXXIXXRXXXXXXXXXXXXXXX

SEQ ID NO: 2 - protein, 44 aa, Homo sapiens
  MISC_FEATURE (44)..(44): Leu residue is capped with an unsubstituted amide moiety
YADAIFTNSYRKVLGQLSARKLLQDIMSRQQGESNQERGARARL

SEQ ID NO: 3 - protein, 44 aa, Artificial sequence; identical to SEQ ID NO: 2
  Amino acid sequence of human GRF

SEQ ID NO: 4 - protein, 29 aa, Homo sapiens
  MISC_FEATURE (29)..(29): Arg residue is capped with an unsubstituted amide moiety
YADAIFTNSYRKVLGQLSARKLLQDIMSR

SEQ ID NO: 5 - protein, 29 aa, Artificial sequence
  Amino acid sequence of minimum active core of human GRF
YADAIFTNSYRKVLGQLSARKLLQDIMSR

SEQ ID NO: 6 - protein, 15 aa, Artificial sequence
  Amino acid sequence corresponding to positions 30 to 44 of human GRF
QQGESNQERGARARL

SEQ ID NO: 7 - protein, 44 aa, Artificial sequence; identical to SEQ ID NO: 2
  Modified GRF peptide

(19) United States
(12) Patent Application Publication
(10) Pub. No.: US 2010/0267636 A1
(43) Pub. Date: Oct. 21, 2010

(54) USE OF CYTOCHROME P450-METABOLIZED DRUGS AND GRF MOLECULES IN COMBINATION THERAPY

(75) Inventor: CHRISTIAN MARSOLAIS, VILLE MONT-ROYAL (CA)

Correspondence Address: FISH & RICHARDSON P.C., P.O. BOX 1022, MINNEAPOLIS, MN, 55440-1022, US

(73) Assignee: THERATECHNOLOGIES INC., MONTREAL (CA)

(21) Appl. No.: 12/764,007

(22) Filed: Apr. 20, 2010

Related U.S. Application Data
(60) Provisional application No. 61/170,852, filed on Apr. 20, 2009.

(51) Int. Cl. A61K 38/16; A61K 31/35; A61P 43/00

(52) U.S. Cl. 514/12; 514/460

(57) ABSTRACT

Combination therapies comprising a drug metabolized by cytochrome P450 and a growth hormone (GH)-inducing compound (such as a GRF molecule) are described, in which there are no or substantially no drug interactions.

### CROSS REFERENCE TO RELATED APPLICATIONS

This application claims the benefit of U.S. Provisional Application Ser. No. 61/170,852, filed Apr. 20, 2009, which is herein incorporated by reference in its entirety.

### REFERENCE TO SEQUENCE LISTING

Pursuant to 37 C.F.R. 1.821(c), a sequence listing is submitted herewith as an ASCII compliant text file named “Sequence_listing.txt” which was created on Apr. 19, 2010 and has a size of 5731 bytes. The content of the aforementioned file named “Sequence_listing.txt” is hereby incorporated by reference in its entirety.

### FIELD OF THE INVENTION

The present invention relates to combination therapy for treatment of conditions. More specifically, the present invention is concerned with combination therapy comprising a plurality of compounds, at least one of which is a drug metabolized by cytochrome P450 and at least another of which is a growth hormone (GH)-inducing compound such as a GRF molecule.

### BACKGROUND OF THE INVENTION

Drug interactions are often observed in cases where multiple drugs are administered to a subject in a combination therapy. Such interactions may alter the pharmacokinetics and clearance of one or more of the drugs being administered, and as such the intended dosage and expected efficacy of a drug may be altered, and in some cases contraindications may occur resulting in potentially serious or life-threatening adverse effects.

Cytochrome P450 (CYP) refers to a large family of proteins involved in diverse enzymatic reactions acting on a variety of substrates. In particular, CYPs are known to be involved in the metabolism of a number of drugs. Changes in CYP activity may affect the metabolism and clearance of various drugs. Given that CYP activity may be influenced by various factors and compounds, CYP-metabolized drugs are often considered as being particularly prone to drug interactions. For example, drug interactions of CYP-metabolized drugs have been reported in GH treatment (Cheung, N. W., et al. (1996), Journal of Clinical Endocrinology and Metabolism, 81(5): 1999-2001; Berglund, E. G., et al. (2002) European Journal of Clinical Investigation, 32: 507-512).

There is therefore a need to identify therapeutic strategies which may be used for treatment of various conditions which are treated with a drug metabolized by cytochrome P450, in which multiple drugs may be used to treat such conditions with minimal drug interactions. For example, it is desirable to identify therapeutic approaches to treat such conditions, which are compatible with CYP-metabolized drugs while avoiding or minimizing drug interactions.

The present description refers to a number of documents, the content of which is herein incorporated by reference in their entirety.

### SUMMARY OF THE INVENTION

The present invention relates to combination therapy for treatment of conditions. More specifically, the present invention is concerned with combination therapy comprising a plurality of compounds, at least one of which is a drug metabolized by cytochrome P450 and at least another of which is a growth hormone (GH)-inducing compound such as a GRF molecule.

More specifically, in accordance with the present invention, there is provided a method of inducing GH levels in a subject undergoing a treatment regimen with or who is a candidate for a treatment regimen with a CYP-metabolized compound, without significantly affecting pharmacokinetics or clearance of said CYP-metabolized compound, said method comprising administering to said subject an effective amount of (hexenoyl trans-3)hGRF(1-44)NH₂.

The invention further provides a method of inducing GH levels in a subject undergoing a treatment regimen with or who is a candidate for a treatment regimen with a CYP-metabolized compound, without modifying said treatment regimen, said method comprising administering to said subject an effective amount of (hexenoyl trans-3)hGRF(1-44)NH₂.

The invention further provides a method of providing GH therapy to a subject undergoing a treatment regimen with or who is a candidate for a treatment regimen with a GYP-metabolized compound, without significantly affecting pharmacokinetics or clearance of said inhibitor, said method comprising administering to said subject an effective amount of (hexenoyl trans-3)hGRF(1-44)NH₂.

The invention further provides a method of providing GH therapy to a subject undergoing a treatment regimen with or who is a candidate for a treatment regimen with a CYP-metabolized compound, without modifying said treatment regimen, said method comprising administering to said subject an effective amount of (hexenoyl trans-3)hGRF(1-44)NH₂.

The invention further provides a method of treating a condition associated with fat accumulation or hypercholesterolemia in a subject undergoing a treatment regimen with or who is a candidate for a treatment regimen with a GYP-metabolized compound, without significantly affecting pharmacokinetics or clearance of said GYP-metabolized compound, said method comprising administering to said subject an effective amount of (hexenoyl trans-3)hGRF(1-44)NH₂.

The invention further provides a method of treating a condition associated with fat accumulation or hypercholesterolemia in a subject undergoing a treatment regimen with or who is a candidate for a treatment regimen with a GYP-metabolized compound, without modifying said treatment regimen, said method comprising administering to said subject an effective amount of (hexenoyl trans-3)hGRF(1-44)NH₂.

The invention further provides a method of treating HIV-associated lipodystrophy in a subject undergoing a treatment regimen with or who is a candidate for a treatment regimen with a CYP-metabolized compound, without significantly affecting pharmacokinetics or clearance of said inhibitor, said method comprising administering to said subject an effective amount of (hexenoyl trans-3)hGRF(1-44)NH₂.

The invention further provides a method of treating excess abdominal fat in a HIV-infected subject with lipodystrophy, wherein said subject is undergoing a treatment regimen with a CYP-metabolized compound or is a candidate for a treatment regimen with a CYP-metabolized compound, without substantially modifying said treatment regimen, said method comprising administering to said subject an effective amount of (hexenoyl trans-3)hGRF(1-44)NH₂.

The invention further provides a method comprising providing information to a subject or to a caregiver of the subject that (hexenoyl trans-3)hGRF(1-44)NH₂ and a CYP-metabolized compound can be co-administered to the subject.

The invention further provides a package comprising: (a) (hexenoyl trans-3)hGRF(1-44)NH₂; and (b) information that (hexenoyl trans-3)hGRF(1-44)NH₂ and a CYP-metabolized compound can be co-administered to a subject.

The invention further provides a use of (hexenoyl trans-3)hGRF(1-44)NH₂ for inducing GH levels in a subject undergoing a treatment regimen with or who is a candidate for a treatment regimen with a CYP-metabolized compound, wherein said use does not significantly affect pharmacokinetics or clearance of said CYP-metabolized compound.

The invention further provides a use of (hexenoyl trans-3)hGRF(1-44)NH₂ for the manufacture of a medicament for inducing GH levels in a subject undergoing a treatment regimen with or who is a candidate for a treatment regimen with a CYP-metabolized compound, wherein said use does not significantly affect pharmacokinetics or clearance of said CYP-metabolized compound.

The invention further provides a use of (hexenoyl trans-3)hGRF(1-44)NH₂ for inducing GH levels in a subject undergoing a treatment regimen with or who is a candidate for a treatment regimen with a CYP-metabolized compound, without modifying said treatment regimen.

The invention further provides a use of (hexenoyl trans-3)hGRF(1-44)NH₂ for the manufacture of a medicament for inducing GH levels in a subject undergoing a treatment regimen with or who is a candidate for a treatment regimen with a CYP-metabolized compound, without modifying said treatment regimen.

The invention further provides a use of (hexenoyl trans-3)hGRF(1-44)NH₂ for providing GH therapy to a subject undergoing a treatment regimen with or who is a candidate for a treatment regimen with a CYP-metabolized compound, wherein said use does not significantly affect pharmacokinetics or clearance of said CYP-metabolized compound.

The invention further provides a use of (hexenoyl trans-3)hGRF(1-44)NH₂ for the manufacture of a medicament for providing GH therapy to a subject undergoing a treatment regimen with or who is a candidate for a treatment regimen with a CYP-metabolized compound, wherein said use does not significantly affect pharmacokinetics or clearance of said CYP-metabolized compound.

The invention further provides a use of (hexenoyl trans-3)hGRF(1-44)NH₂ for providing GH therapy to a subject undergoing a treatment regimen with or who is a candidate for a treatment regimen with a CYP-metabolized compound, without modifying said treatment regimen.

The invention further provides a use of (hexenoyl trans-3)hGRF(1-44)NH₂ for the manufacture of a medicament for providing GH therapy to a subject undergoing a treatment regimen with or who is a candidate for a treatment regimen with a CYP-metabolized compound, without modifying said treatment regimen.

The invention further provides a use of (hexenoyl trans-3)hGRF(1-44)NH₂ for treating a condition associated with fat accumulation or hypercholesterolemia in a subject undergoing a treatment regimen with or who is a candidate for a treatment regimen with a CYP-metabolized compound, wherein said use does not significantly affect pharmacokinetics or clearance of said CYP-metabolized compound.

The invention further provides a use of (hexenoyl trans-3)hGRF(1-44)NH₂ for the manufacture of a medicament for treating a condition associated with fat accumulation or hypercholesterolemia in a subject undergoing a treatment regimen with or who is a candidate for a treatment regimen with a CYP-metabolized compound, wherein said use does not significantly affect pharmacokinetics or clearance of said CYP-metabolized compound.

The invention further provides a use of (hexenoyl trans-3)hGRF(1-44)NH₂ for treating a condition associated with fat accumulation or hypercholesterolemia in a subject undergoing a treatment regimen with or who is a candidate for a treatment regimen with a CYP-metabolized compound, without modifying said treatment regimen.

The invention further provides a use of (hexenoyl trans-3)hGRF(1-44)NH₂ for the manufacture of a medicament for treating a condition associated with fat accumulation or hypercholesterolemia in a subject undergoing a treatment regimen with or who is a candidate for a treatment regimen with a CYP-metabolized compound, without modifying said treatment regimen.

The invention further provides a use of (hexenoyl trans-3)hGRF(1-44)NH₂ for treating excess abdominal fat in a HIV-infected subject with lipodystrophy, wherein said subject is undergoing a treatment regimen with a CYP-metabolized compound or is a candidate for a treatment regimen with a CYP-metabolized compound, wherein said use does not significantly affect pharmacokinetics or clearance of said CYP-metabolized compound.

The invention further provides a use of (hexenoyl trans-3)hGRF(1-44)NH₂ for the manufacture of a medicament for treating excess abdominal fat in a HIV-infected subject with lipodystrophy, wherein said subject is undergoing a treatment regimen with a CYP-metabolized compound or is a candidate for a treatment regimen with a CYP-metabolized compound, wherein said use does not significantly affect pharmacokinetics or clearance of said CYP-metabolized compound.

The invention further provides a use of (hexenoyl trans-3)hGRF(1-44)NH₂ for treating excess abdominal fat in a HIV-infected subject with lipodystrophy, wherein said subject is undergoing a treatment regimen with a CYP-metabolized compound or is a candidate for a treatment regimen with a CYP-metabolized compound, without modifying said treatment regimen.

The invention further provides a use of (hexenoyl trans-3)hGRF(1-44)NH₂ for the manufacture of a medicament for treating excess abdominal fat in a HIV-infected subject with lipodystrophy, wherein said subject is undergoing a treatment regimen with a CYP-metabolized compound or is a candidate for a treatment regimen with a CYP-metabolized compound, without modifying said treatment regimen.

The invention further provides (hexenoyl trans-3)hGRF(1-44)NH₂ for inducing GH levels in a subject undergoing a treatment regimen with or who is a candidate for a treatment regimen with a CYP-metabolized compound, wherein said (hexenoyl trans-3)hGRF(1-44)NH₂ does not significantly affect pharmacokinetics or clearance of said CYP-metabolized compound.

The invention further provides (hexenoyl trans-3)hGRF(1-44)NH₂ for the manufacture of a medicament for inducing GH levels in a subject undergoing a treatment regimen with or who is a candidate for a treatment regimen with a CYP-metabolized compound, wherein said (hexenoyl trans-3)hGRF(1-44)NH₂ does not significantly affect pharmacokinetics or clearance of said CYP-metabolized compound.

The invention further provides (hexenoyl trans-3)hGRF(1-44)NH₂ for inducing GH levels in a subject undergoing a treatment regimen with or who is a candidate for a treatment regimen with a CYP-metabolized compound, without modifying said treatment regimen.

The invention further provides (hexenoyl trans-3)hGRF(1-44)NH₂ for the manufacture of a medicament for inducing GH levels in a subject undergoing a treatment regimen with or who is a candidate for a treatment regimen with a CYP-metabolized compound, without modifying said treatment regimen.

The invention further provides (hexenoyl trans-3)hGRF(1-44)NH₂ for providing GH therapy to a subject undergoing a treatment regimen with or who is a candidate for a treatment regimen with a CYP-metabolized compound, wherein said (hexenoyl trans-3)hGRF(1-44)NH₂ does not significantly affect pharmacokinetics or clearance of said CYP-metabolized compound.

The invention further provides (hexenoyl trans-3)hGRF(1-44)NH₂ for the manufacture of a medicament for providing GH therapy to a subject undergoing a treatment regimen with or who is a candidate for a treatment regimen with a CYP-metabolized compound, wherein said (hexenoyl trans-3)hGRF(1-44)NH₂ does not significantly affect pharmacokinetics or clearance of said CYP-metabolized compound.

The invention further provides (hexenoyl trans-3)hGRF(1-44)NH₂ for providing GH therapy to a subject undergoing a treatment regimen with or who is a candidate for a treatment regimen with a CYP-metabolized compound, without modifying said treatment regimen.

The invention further provides (hexenoyl trans-3)hGRF(1-44)NH₂ for the manufacture of a medicament for providing GH therapy to a subject undergoing a treatment regimen with or who is a candidate for a treatment regimen with a CYP-metabolized compound, without modifying said treatment regimen.

The invention further provides (hexenoyl trans-3)hGRF(1-44)NH₂ for treating a condition associated with fat accumulation or hypercholesterolemia in a subject undergoing a treatment regimen with or who is a candidate for a treatment regimen with a CYP-metabolized compound, wherein said (hexenoyl trans-3)hGRF(1-44)NH₂ does not significantly affect pharmacokinetics or clearance of said CYP-metabolized compound.

The invention further provides (hexenoyl trans-3)hGRF(1-44)NH₂ for the manufacture of a medicament for treating a condition associated with fat accumulation or hypercholesterolemia in a subject undergoing a treatment regimen with or who is a candidate for a treatment regimen with a CYP-metabolized compound, wherein said (hexenoyl trans-3)hGRF(1-44)NH₂ does not significantly affect pharmacokinetics or clearance of said CYP-metabolized compound.

The invention further provides (hexenoyl trans-3)hGRF(1-44)NH₂ for treating a condition associated with fat accumulation or hypercholesterolemia in a subject undergoing a treatment regimen with or who is a candidate for a treatment regimen with a CYP-metabolized compound, without modifying said treatment regimen.

The invention further provides (hexenoyl trans-3)hGRF(1-44)NH₂ for the manufacture of a medicament for treating a condition associated with fat accumulation or hypercholesterolemia in a subject undergoing a treatment regimen with or who is a candidate for a treatment regimen with a CYP-metabolized compound, without modifying said treatment regimen.

The invention further provides (hexenoyl trans-3)hGRF(1-44)NH₂ for treating excess abdominal fat in a HIV-infected subject with lipodystrophy, wherein said subject is undergoing a treatment regimen with a CYP-metabolized compound or is a candidate for a treatment regimen with a CYP-metabolized compound, wherein said (hexenoyl trans-3)hGRF(1-44)NH₂ does not significantly affect pharmacokinetics or clearance of said CYP-metabolized compound.

The invention further provides (hexenoyl trans-3)hGRF(1-44)NH₂ for the manufacture of a medicament for treating excess abdominal fat in a HIV-infected subject with lipodystrophy, wherein said subject is undergoing a treatment regimen with a CYP-metabolized compound or is a candidate for a treatment regimen with a CYP-metabolized compound, wherein said (hexenoyl trans-3)hGRF(1-44)NH₂ does not significantly affect pharmacokinetics or clearance of said CYP-metabolized compound.

The invention further provides (hexenoyl trans-3)hGRF(1-44)NH₂ for treating excess abdominal fat in a HIV-infected subject with lipodystrophy, wherein said subject is undergoing a treatment regimen with a GYP-metabolized compound or is a candidate for a treatment regimen with a CYP-metabolized compound, without modifying said treatment regimen.

The invention further provides (hexenoyl trans-3)hGRF(1-44)NH₂ for the manufacture of a medicament for treating excess abdominal fat in a HIV-infected subject with lipodystrophy, wherein said subject is undergoing a treatment regimen with a CYP-metabolized compound or is a candidate for a treatment regimen with a GYP-metabolized compound, without modifying said treatment regimen.

The invention further provides a composition comprising (hexenoyl trans-3)hGRF(1-44)NH₂ and a pharmaceutically acceptable excipient for inducing GH levels in a subject undergoing a treatment regimen with or who is a candidate for a treatment regimen with a GYP-metabolized compound, wherein said (hexenoyl trans-3)hGRF(1-44)NH₂ does not significantly affect pharmacokinetics or clearance of said CYP-metabolized compound.

The invention further provides a composition comprising (hexenoyl trans-3)hGRF(1-44)NH₂ and a pharmaceutically acceptable excipient for inducing GH levels in a subject undergoing a treatment regimen with or who is a candidate for a treatment regimen with a CYP-metabolized compound, without modifying said treatment regimen.

The invention further provides a composition comprising (hexenoyl trans-3)hGRF(1-44)NH₂ and a pharmaceutically acceptable excipient for providing GH therapy to a subject undergoing a treatment regimen with or who is a candidate for a treatment regimen with a CYP-metabolized compound, wherein said (hexenoyl trans-3)hGRF(1-44)NH₂ does not significantly affect pharmacokinetics or clearance of said CYP-metabolized compound.

The invention further provides a composition comprising (hexenoyl trans-3)hGRF(1-44)NH₂ and a pharmaceutically acceptable excipient for providing GH therapy to a subject undergoing a treatment regimen with or who is a candidate for a treatment regimen with a CYP-metabolized compound, without modifying said treatment regimen.

The invention further provides a composition comprising (hexenoyl trans-3)hGRF(1-44)NH₂ and a pharmaceutically acceptable excipient for treating a condition associated with fat accumulation or hypercholesterolemia in a subject undergoing a treatment regimen with or who is a candidate for a treatment regimen with a CYP-metabolized compound, wherein said (hexenoyl trans-3)hGRF(1-44)NH₂ does not significantly affect pharmacokinetics or clearance of said CYP-metabolized compound.

The invention further provides a composition comprising (hexenoyl trans-3)hGRF(1-44)NH₂ and a pharmaceutically acceptable excipient for treating a condition associated with fat accumulation or hypercholesterolemia in a subject undergoing a treatment regimen with or who is a candidate for a treatment regimen with a CYP-metabolized compound, without modifying said treatment regimen.

The invention further provides a composition comprising (hexenoyl trans-3)hGRF(1-44)NH₂ and a pharmaceutically acceptable excipient for treating HIV-associated lipodystrophy in a subject undergoing a treatment regimen with or who is a candidate for a treatment regimen with a CYP-metabolized compound, wherein said (hexenoyl trans-3)hGRF(1-44)NH₂ does not significantly affect pharmacokinetics or clearance of said CYP-metabolized compound.

The invention further provides a composition comprising (hexenoyl trans-3)hGRF(1-44)NH₂ and a pharmaceutically acceptable excipient for treating HIV-associated lipodystrophy in a subject undergoing a treatment regimen with or who is a candidate for a treatment regimen with a CYP-metabolized compound, without modifying said treatment regimen.

In an embodiment, the above-mentioned method, use, (hexenoyl trans-3)hGRF(1-44)NH₂ or composition further comprises, prior to said treating, selecting a subject who is undergoing or who is a candidate for a treatment regimen with a CYF-metabolized compound.

In an embodiment, the above-mentioned method further comprises providing information to the subject or to the caregiver of the subject that (hexenoyl trans-3)hGRF(1-44)NH₂ can be co-administered to the subject without affecting pharmacokinetics of said CYP-metabolized compound. In another embodiment, the above-mentioned method further comprises providing information to the subject or to the caregiver of the subject that (hexenoyl trans-3)hGRF(1-44)NH₂ can be co-administered to the subject without modifying the treatment regimen of said CYP-metabolized compound.

In an embodiment, the above-mentioned CYP-metabolized compound is a statin. In a further embodiment, the above-mentioned statin is simvastatin.

In an embodiment, the (hexenoyl trans-3)hGRF(1-44)NH₂ is administered or is adapted for administration at a daily dose of 2 mg.

In an embodiment, the (hexenoyl trans-3)hGRF(1-44)NH₂ is administered subcutaneously or is adapted for subcutaneous administration.

Other objects, advantages and features of the present invention will become more apparent upon reading of the following non-restrictive description of specific embodiments thereof, given by way of example only with reference to the accompanying drawings.

### BRIEF DESCRIPTION OF THE DRAWINGS

In the appended drawings:

FIG. 1a shows concentration-time profiles of simvastatin with (Treatment A) and without (Treatment B) pre-treatment with (hexenoyl trans-3)hGRF(1-44)NH₂; and

FIG. 1b shows concentration-time profiles of simvastatin acid with (Treatment A) and without (Treatment B) pre-treatment with (hexenoyl trans-3)hGRF(1-44)NH₂.

### DESCRIPTION OF ILLUSTRATIVE EMBODIMENTS

The present invention relates to combination therapies utilizing a CYP-metabolized compound. In an embodiment, the present invention relates to a combined therapy comprising a CYP-metabolized compound (e.g., a statin such as simvastatin) and a growth hormone releasing molecule, such as a growth hormone releasing factor (GRF) or a GRF analog (e.g., (hexenoyl trans-3)hGRF(1-44)NH₂, also referred to as TH9507 herein). In embodiments, such therapies relate to the treatment of a condition associated with fat accumulation, fat redistribution and/or elevated cholesterol, such as HIV-associated lipodystrophy and hypercholesterolemia.

In embodiments, the invention relates to a combination therapy comprising administering an effective amount of GRF or an analog thereof to a subject who is undergoing treatment with a CYP-metabolized compound or who is a candidate for treatment with a CYP-metabolized compound.

Growth hormone (GH) or somatotropin is secreted by the pituitary gland. Its activity is fundamental for the linear growth of a young organism but also for the maintenance of the integrity at its adult state. GH acts directly or indirectly on the peripheral organs by stimulating the synthesis of growth factors (insulin-like growth factor-I or IGF-I) or of their receptors (epidermal growth factor or EGF). The direct action of GH is of the type referred to as anti-insulinic, which favors the lipolysis at the level of adipose tissues. Through its action on IGF-I (somatomedin C) synthesis and secretion, GH stimulates the growth of cartilage and the bones (structural growth), protein synthesis and cellular proliferation in multiple peripheral organs, including muscle and skin. In adults, GH participates in the maintenance of a protein anabolism state and plays a primary role in the tissue regeneration phenomenon after a trauma. GH has also been shown to modulate the expression and/or activity of cytochrome P450 in animal models and in humans (Morgan et al., J Biol Chem 260:11895-8); Waxman et al., Mol Endocrinol. 4: 447-454; Jarukamjorn et al., Toxicology, 219(1-3): 97-105; Liddle et al., Arch Biochem Biophys 298: 159-166; Liddle et al., Journal of Clinical Endocrinology & Metabolism 83(7): 2411-2416; Cheung, N. W., et al., (1996), Journal of Clinical Endocrinology and Metabolism, 81(5): 1999-2001; Robertson et al., Biochem Biophys Res Commun. 242(1): 57-60)

The secretion of GH by the pituitary gland is principally controlled by two hypothalamic peptides, somatostatin and growth hormone-releasing hormone (GHRH; also known as growth hormone-releasing factor or GRF). Somatostatin inhibits its secretion, whereas GRF stimulates it.

Among all known GRF molecules, GRF analogs containing a hydrophobic tail as defined in the present application consist of modified versions or analogs of human GRF that have been shown to have higher proteolytic stability in biological milieu and as a result, these analogs were shown to display longer duration of action resulting in enhanced growth hormone secretion and insulin like growth factor-1 synthesis (U.S. Pat. Nos. 5,861,379 and 5,939,386). Due to their superior plasma stability and pharmacological properties compared to the native GRF (1-44) amide, these GRF analogs were shown to confer therapeutic efficacy in several medical conditions, e.g., wasting associated with COPD (International Application No. WO 05/037307), recovery after hip fracture, frailty in elderly population, enhancing immune response and HIV-associated lipodystrophy (U.S. Pat. No. 7,316,997).

The term “GRF molecule” as used in the context of the present invention includes, without limitation, human native GRF (amino acids 1-44, SEQ ID NO: 3) and fragments (1-40), (1-29, [SEQ ID NO: 5]), fragments ranging between 1-29 and the 1-44 sequence, and any other fragments; GRF from other species and fragments thereof; GRF variants containing amino acid(s) substitution(s), addition(s) and/or deletion(s) such that the amino acid sequence of the variant has at least about 90% of homology with the native amino acid sequence, in an embodiment at least about 95% of homology with the native amino acid sequence. In an embodiment, the above-mentioned fragments/variants retain at least about 10% of the activity of stimulating GH secretion as compared to the native GRF; derivatives or analogs of GRF or fragments or variants thereof having for a example an organic group or a moiety coupled to the GRF amino acid sequence at the N-terminus, the C-terminus or on the side-chain (e.g., human native GRF having a C-terminal unsubstituted amide moiety, SEQ ID NO: 2; fragment 1-29 of human native GRF having a C-terminal unsubstituted amide moiety, SEQ ID NO: 4); and salts of GRF (human or from other species), as well as salts of GRF fragments, variants, analogs and derivatives. The GRF molecules of the present invention also encompass the GRF molecules currently known in the art, including, without limitation, the albumin-conjugated GRF (U.S. Pat. No. 7,268,113); pegylated GRF peptide (U.S. Pat. Nos. 7,256,258 and 6,528,485); porcine GRF (1-40) (U.S. Pat. No. 6,551,996); canine GRF (U.S. patent application no. 2005/0064554); GRF variants of 1-29 to 1-44 amino acid length (U.S. Pat. Nos. 5,846,936, 5,696,089, 5,756,458 and 5,416,073, and U.S. patent application Nos. 2006/0128615 and 2004/0192593); and Pro⁰-GRF peptide and variants thereof (U.S. Pat. No. 5,137,872).

The GRF analogs include those described in U.S. Pat. Nos. 5,681,379 and 5,939,386, which also describe their method of synthesis. More particularly, these GRF analogs are defined by the following formula A:

X-GRF Peptide (A)

The GRF peptide is a peptide of the following formula B:

(SEQ ID NO: 1)

Al-A2-Asp-Ala-Ile-Phe-Thr-A8-Ser-Tyr-Arg-Lys-A13-
Leu-A15-Gln-Leu-A18-Ala-Arg-Lys-Leu-Leu-A24-A25-
Ile-A27-A28-Arg-A30-R0 (B)

wherein,
A1 is Tyr or His;
A2 is Val or Ala;
A8 is Asn or Ser;
A13 is Val or Ile;
A15 is Ala or Gly;
A18 is Ser or Tyr;
A24 is Gln or His;
A25 is Asp or Glu;
A27 is Met, Ile or Nle
A28 is Ser or Asn;
A30 is a bond or amino acid sequence of 1 up to 15 residues; and
R0 is NH₂ or NH—(CH₂)ₙ—CONH₂, with n=1 to 12.
   wherein X is:
   (a) a hydrophobic tail anchored via an amide bond to the N-terminus of the peptide, said hydrophobic tail comprising (i) a backbone of 5 to 7 atoms;
   wherein said backbone can be substituted by C₁₋₆ alkyl, C₃₋₆ cycloalkyl, or C₆₋₁₂ aryl, and
   (ii) at least one rigidifying moiety connected to at least two atoms of the backbone; the rigidifying moiety being a double bond, a triple bond, a saturated or unsaturated C₃₋₉ cycloalkyl, or a C₆₋₁₂ aryl; or
   (b) a moiety selected from:

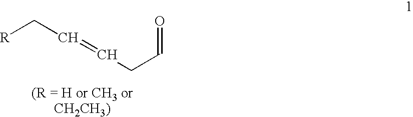

cis or trans,

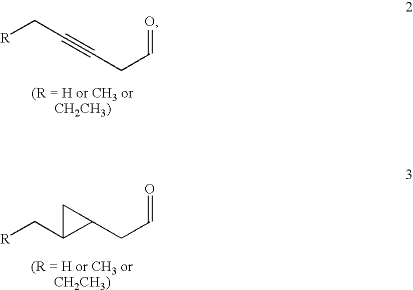

cis or trans, both as racemic mixtures
or pure enantiomeric pairs,

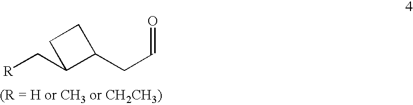

cis or trans, both as racemic mixtures
or pure enantiomeric pairs,

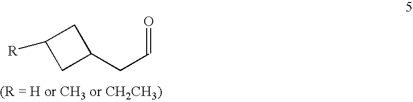

cis or trans, (when H),

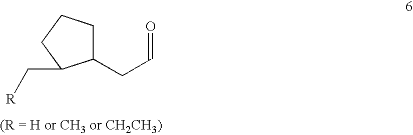

cis or trans, both as racemic mixtures
or pure enantiomeric pairs,

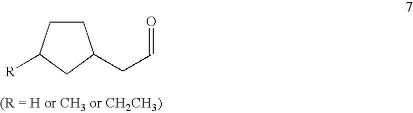

cis or trans, (when R≠H)
both as racemic mixtures
or pure enantiomeric pairs,

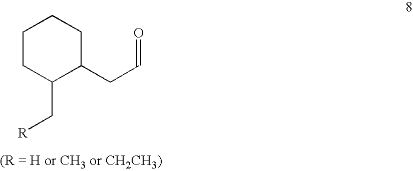

cis or trans, both as racemic mixtures
or pure enantiomeric pairs,

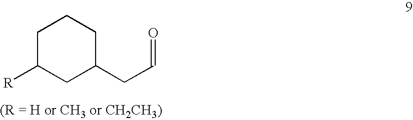

cis or trans, (when R≠H)
both as racemic mixtures
or pure enantiomeric pairs,

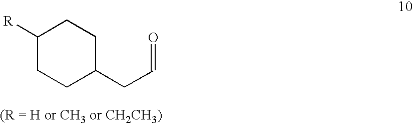

cis or trans, (when R≠H),

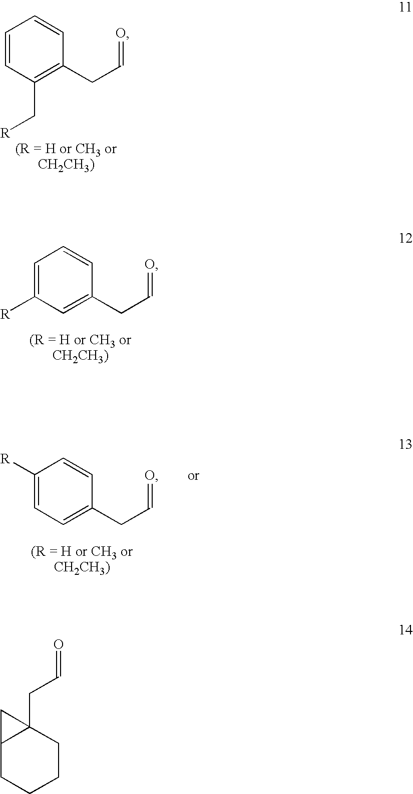

In an embodiment, group X is:

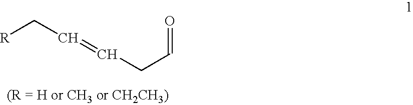

cis or trans,

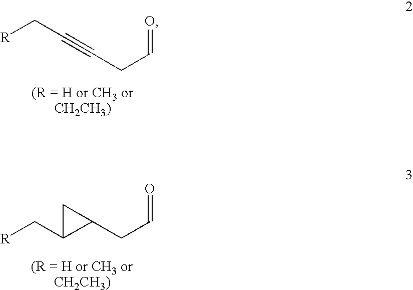

cis or trans, both as racemic mixtures
or pure enantiomeric pairs,

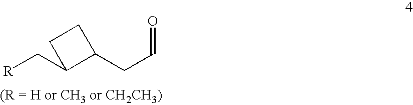

cis or trans, both as racemic mixtures
or pure enantiomeric pairs,

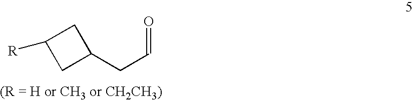

cis or trans, (when R≠H),

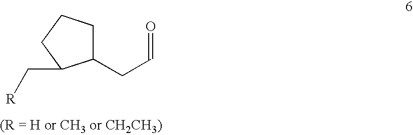

cis or trans, both as racemic mixtures
or pure enantiomeric pairs,

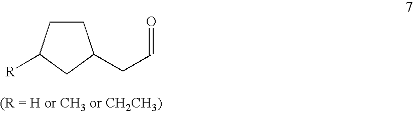

cis or trans, (when R≠H)
both as racemic mixtures
or pure enantiomeric pairs,

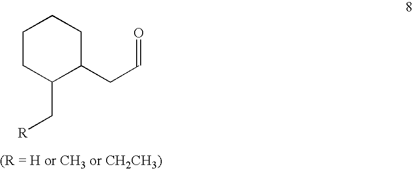

cis or trans, both as racemic mixtures
or pure enantiomeric pairs,

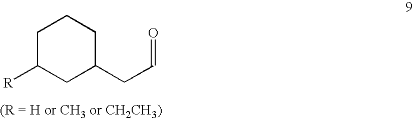

cis or trans, (when R≠H)
both as racemic mixtures
or pure enantiomeric pairs,

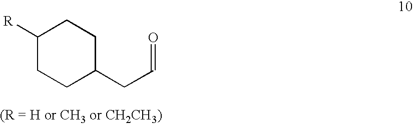

cis or trans, (when R≠H),

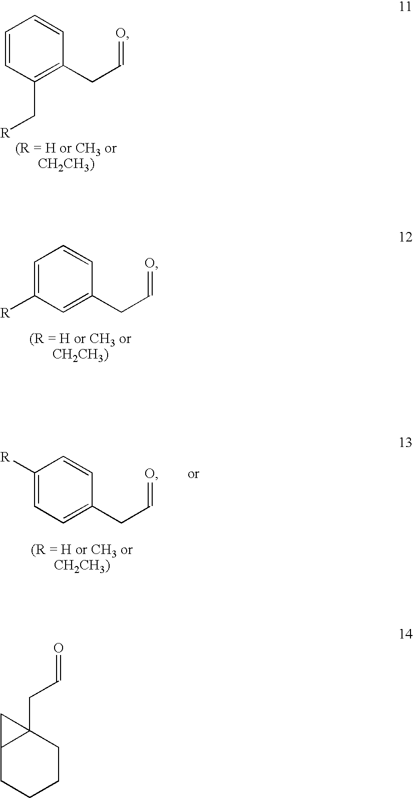

In an embodiment, in formula B, A30 is:
(a) a bond,
(b) an amino acid sequence corresponding to positions 30-44 of a natural GRF peptide (e.g., positions 30-44 of human GRF peptide, SEQ ID NO: 6), or
(c) the amino acid sequence of (b) having a 1-14 amino acid deletion from its C-terminus.

In an embodiment, the GRF molecule is (hexenoyl trans-3)hGRF(1-44)NH₂ (also referred to as (trans-3-hexenoyl)hGRF (1-44) amide or TH9507 herein). (hexenoyl trans-3)hGRF(1-44)NH₂) is a synthetic human growth hormone releasing factor analog that comprises the 44-amino acid sequence of human growth hormone releasing factor (hGRF) on which a hexenoyl moiety, a C₆ side chain, has been anchored on Tyr1 at the N-terminus.

(trans-3-hexenoyl)hGRF (1-44)NH₂ has the following structure (SEQ ID NO: 7): (trans)CH₃—CH₂—CH═CH—CH₂—CO-Tyr-Ala-Asp-Ala-Ile-Phe-Thr-Asn-Ser-Tyr-Arg-Lys-Val-Leu-Gly-Gln-Leu-Ser-Ala-Arg-Lys-Leu-Leu-Gln-Asp-Ile-Met-Ser-Arg-Gln-Gln-Gly-Glu-Ser-Asn-Gln-Glu-Arg-Gly-Ala-Arg-Ala-Arg-Leu-NH₂.

“GH therapy” as used herein refers to treatment which results in an increase in GH levels in a subject. In an embodiment, the subject may exhibit a GH deficiency (i.e., lower than normal levels of GH) and therefore such GH therapy is effected to increase GH levels with a view to reverse such deficiency. In a further embodiment, the subject may exhibit normal GH levels and therefore such GH therapy is effected to increase GH levels to result in higher than normal GH levels. GH therapy may in embodiments be achieved by administration of GH or a fragment, variant or analog thereof, and in further embodiments may be achieved by administering a compound which induces GH secretion, e.g., a GH secretagogue, GRF, or a GRF molecule. “Compound which induces GH secretion” as used herein refers to any compound or molecule, natural or synthetic, which may result in, either directly or indirectly, GH secretion and/or an increase in GH secretion. In an embodiment, the compound which induces GH secretion is a GRF molecule.

In embodiments, a GRF molecule may be used to treat a condition such as HIV-associated lipodystrophy, HIV-lipohypertrophy, abdominal obesity, GH deficiency, frailty, mild cognitive impairment, immune deficiency, wasting associated with a chronic condition or long-term condition, or malnutrition associated with a chronic condition or a long-term condition. Chronic conditions include, without limitation, HIV infection, AIDS, cystic fibrosis, chronic obstructive pulmonary disease (COPD), hip fracture, trauma, and major surgery.

In further embodiments, a GRF molecule may be used to treat a condition associated with fat accumulation. Fat accumulation is observed in a range of conditions or syndromes such as obesity, metabolic syndrome (also known as syndrome X), and excess abdominal fat in a HIV-infected subject with lipodystrophy. All these conditions include features which are known to increase the risk of diabetes and/or cardiovascular diseases.

“CYP-metabolized compound” as used herein refers to any compound whose pharmacokinetics and/or clearance is affected by one or more components/activities of the CYP system. In an embodiment, such a compound is degraded as a result of CYP activity, and therefore in embodiments, increases in and/or induction of CYP activity results in increased degradation and in turn increased clearance of the compound whereas decreases in and/or inhibition of CYP activity results in increased plasma levels of such a compound. In further embodiments, such a compound is metabolized to an active form as a result of CYP activity, and therefore in embodiments, increases in and/or induction of CYP activity results in increased plasma levels of the active form of the compound whereas decreases in and/or inhibition of CYP activity results in decreased plasma levels of the active form of the compound. In an embodiment, the CYP-metabolized compound is a drug. Table I provides examples of drugs known to be metabolized by one or more isozymes of the CYP system.

Table I: Drugs known to be metabolized by one or more isozymes of the CYP 3A family (including CYP3A4, 5 and 7) system (adapted from Flockhart DA. Drug Interactions: Cytochrome P450 Drug Interaction Table. Version 5.0, Indiana University School of Medicine (2007). http://medicine.iupui.edu/clinpharm/ddis/table.asp.

| Macrolide antibiotics: | Anti-arrhythmics: | Benzodiazepines: |
|---|---|---|
| clarithromycin | quinidine→ | alprazolam |
| erythromycin (not 3A5) | 3-OH (not 3A5) | diazepam |
| NOT azithromycin | | → 3OH |
| telithromycin | | midazolam |
| | | triazolam |
| Immune Modulators: | HIV Antivirals: | Prokinetics: |
| cyclosporine | indinavir | cisapride |
| tacrolimus (FK506) | ritonavir | |
| | saquinavir | |
| Antihistamines: | Calcium Channel | HMG-CoA Reductase |
| astemizole | Blockers: | Inhibitors: |
| chlorpheni-ramine | amlodipine | atorvastatin |
| | diltiazem | lovastatin |
| | felodipine | NOT pravasta-tin |
| | nifedipine | NOT rosu-vastatin |
| | nisoldipine | simvastatin |
| | nitrendipine | |
| | verapamil | |
| Others: | | |
| aripiprazole | | |
| buspirone | | |
| gleevec | | |
| haloperidol | | |
| methadone | | |
| pimozide | | |
| quinine | | |
| sildenafil | | |
| tamoxifen | | |
| trazodone | | |
| vincristine | | |

In an embodiment, the CYP-metabolized drug is metabolized by an isoenzyme of the CYP3A family, in a further embodiment the CYP3A4 isoenzyme.

In an embodiment, the CYP-metabolized drug is a statin. The term “statin” refers to a class of drug used to lower plasma cholesterol levels by inhibiting an enzyme involved in cholesterol synthesis known as HMG-CoA reductase. In an embodiment, the CYP-metabolized drug is a statin metabolized by the CYP3A4 isoenzyme, such as atorvastatin (Lipitor™), lovastatin (Mevacor™, Altocor™, Altoprev™) and simvastatin (Zocor™, Lipex™) (Talbert R L, J Am Pharm Assoc 2006; 46: 479-490). In a further embodiment, the statin is simvastatin.

“Pharmacokinetics” as used herein refers to the parameters of absorption and distribution of an administered drug, such as the rate at which a drug action begins and the duration of the effect, the concentration of the drug in tissues, organs, body fluids (e.g., blood, plasma or urine concentration), the chemical modifications of the substance in the body (e.g., by liver enzymes, such as the GYP system) and the effects and routes of excretion of the metabolites of the drug. In an embodiment, the pharmacokinetics comprises the blood or plasma concentration.

The expression “without modifying said treatment regimen” means that the administration of the GRF molecule (e.g., (hexenoyl trans-3)hGRF(1-44)NH₂) to the subject does not require any modifications to the CYP-metabolized compound-based treatment regimen, i.e. there is no need to:
stop or temporarily delay CYP-metabolized compound-based treatment regimen;
replace the CYP-metabolized compound administered with another CYP-metabolized compound or different drug;
change the dosage, or change the frequency of administration of the CYP-metabolized compound; and/or
monitor possible drug interactions and/or changes in the efficacy of the treatment.

In embodiments, the invention provides a combination therapy comprising a use of (a) a GRF molecule and a CYP-metabolized compound; (b) a composition comprising a GRF molecule and a pharmaceutically acceptable carrier and a composition comprising a CYP-metabolized compound and a pharmaceutically acceptable carrier; or (c) a composition comprising a GRF molecule, a GYP-metabolized compound, and in an embodiment further comprising a pharmaceutically acceptable carrier.

“Combination therapy” as used herein refers to administration of two or more compounds or compositions to a subject, for example a GRF molecule or a composition comprising a GRF molecule, and a CYP-metabolized compound or a composition comprising a CYP-metabolized compound. In embodiments, the combination therapy may be administered sequentially or simultaneously. For example, in an embodiment the GRF molecule or composition comprising the GRF molecule may be administered to a subject undergoing treatment with a CYP-metabolized compound, i.e., to which a CYP-metabolized compound has already been administered. In a further embodiment, the GRF molecule or composition comprising the GRF molecule may be administered to a subject who is a candidate for treatment with a CYP-metabolized compound, i.e., a subject who has been identified as one who may benefit from treatment with a CYP-metabolized compound, and thus to which a CYP-metabolized compound may be administered at a later time. In a further embodiment the GRF molecule and CYP-metabolized compound (or compositions thereof) may be administered at substantially the same time, either via separate administration or administered together in the same composition.

As noted above, in various embodiments, the above-mentioned GRF molecule and CYP-metabolized compound may be used therapeutically in compositions, formulations or medicaments to effect the above-noted combination therapy or to prevent or treat the above-noted conditions. The invention provides corresponding methods of medical treatment, in which a therapeutic dose of a GRF molecule and/or a CYP-metabolized compound is administered in a pharmacologically acceptable formulation(s), e.g., to a subject or subject in need thereof. Accordingly, the invention also provides therapeutic compositions comprising a GRF molecule and/or a CYP-metabolized compound and a pharmacologically acceptable excipient or carrier. In an embodiment, such compositions include the GRF molecule and/or CYP-metabolized compound in a therapeutically or prophylactically effective amount sufficient to effect the above-noted combined therapy and to prevent or treat the above-noted conditions. The composition may be soluble in an aqueous solution at a physiologically acceptable pH.

A “therapeutically effective amount” refers to an amount effective, at dosages and for periods of time necessary, to achieve the desired therapeutic result, such as to effect the above-noted combination therapy or to prevent or treat the above-noted conditions, in a subject in need thereof. A therapeutically effective amount of a GRF molecule or a CYP-metabolized compound may vary according to factors such as the disease state, age, sex, and weight of the individual, and the ability of the compound to elicit a desired response in the individual. Dosage regimens may be adjusted to provide the optimum therapeutic response. A therapeutically effective amount is also one in which any toxic or detrimental effects of the compound are outweighed by the therapeutically beneficial effects. A “prophylactically effective amount” refers to an amount effective, at dosages and for periods of time necessary, to achieve the desired prophylactic result, such as preventing or inhibiting the rate of onset or progression of the above-noted conditions. A prophylactically effective amount can be determined as described above for the therapeutically effective amount. For any particular subject, specific dosage regimens may be adjusted over time according to the individual need and the professional judgement of the person administering or supervising the administration of the compositions.

As used herein “pharmaceutically acceptable carrier” or “excipient” includes any and all solvents, diluents, pH modifying agents, dispersion media, coatings, antibacterial and antifungal agents, isotonic and absorption delaying agents, and the like that are physiologically compatible. In one embodiment, the carrier is suitable for parenteral administration. Alternatively, the carrier can be suitable for intravenous, intraperitoneal, intramuscular, subcutaneous, sublingual or oral administration. Pharmaceutically acceptable carriers include sterile aqueous solutions or dispersions and sterile powders for the preparation of sterile injectable solutions or dispersion. The use of such media and agents for pharmaceutically active substances is well known in the art. Except insofar as any conventional media or agent is incompatible with the active compound, use thereof in the pharmaceutical compositions of the invention is contemplated.

Therapeutic compositions typically must be sterile and stable under the conditions of manufacture and storage. The composition can be formulated as a solution, microemulsion, liposome, or other ordered structure suitable to high drug concentration. Moreover, a GRF molecule or CYP-metabolized compound can be administered in a time release formulation (e.g., sustained release, controlled release, delayed release). The active compounds can be prepared with carriers that will protect the compound against rapid release, such as a controlled release formulation. Many methods for the preparation of such formulations are generally known to those skilled in the art.

Sterile injectable solutions can be prepared by incorporating the active compound (e.g. a GRF molecule or CYP-metabolized compound) in the required amount in an appropriate solvent with one or a combination of excipients, as required, followed by filtered sterilization. Generally, dispersions are prepared by incorporating the active compound into a sterile vehicle which contains a basic dispersion medium and other excipient(s). In the case of sterile powders for the preparation of sterile injectable solutions, the preferred methods of preparation are vacuum drying and freeze-drying (lyophilization) which yields a powder of the active ingredient plus any additional desired ingredient from a previously sterile-filtered solution thereof. In accordance with an alternative aspect of the invention, a GRF molecule or CYP-metabolized compound may be formulated with one or more additional compounds that enhance its solubility.

In accordance with another aspect of the invention, therapeutic compositions of the present invention, comprising a GRF molecule and/or CYP-metabolized compound, may be provided in containers, kits or packages (e.g., commercial packages) which further comprise instructions for its use for the above-noted combination therapy or to prevent or treat the above-noted conditions.

Accordingly, the invention further provides a package comprising a GRF molecule or the above-mentioned composition comprising a GRF molecule together with instructions to the effect that the GRF molecule or the above-mentioned composition comprising a GRF molecule is suitable for combination therapy with a CYP-metabolized compound, i.e., that it may be administered to a subject undergoing treatment with or who is a candidate for treatment with a CYP-metabolized compound. The kit or package may further comprise containers, buffers (e.g., to resuspend the compound(s), devices for administering the compound(s), etc.

“Suitable for combination therapy” in the present context refers to no or substantially no drug interactions between a GRF molecule and CYP-metabolized compound. In an embodiment, a GRF molecule and a CYP-metabolized compound are suitable for combination therapy if the pharmacokinetics of the compounds is not significantly affected/modulated in the presence (relative to the absence) of the other compound. In an embodiment, a GRF molecule and a CYP-metabolized compound are suitable for combination therapy if one or more pharmacokinetics parameters of the compounds is/are not affected/modulated by more than about 20% in the presence of the other compound (relative to the absence thereof). In an embodiment, one or more pharmacokinetic parameters of a compound are not “significantly affected” by the presence of another compound If they are not modulated by more than about 20% in the presence (relative to the absence) of the other compound. In an embodiment, the one or more pharmacokinetics parameters comprise the blood or plasma concentration of the compound. Methods for determining/analyzing pharmacokinetics parameters are well known in the art (see, for example, Thomas N. Tozer and Malcolm Rowland, Introduction to Pharmacokinetics and Pharmacodynamics: The Quantitative Basis of Drug Therapy, Lippincott Williams & Wilkins, 2006; Malcolm Rowland and Thomas N. Tozer, Clinical pharmacokinetics: concepts and applications, Williams & Wilkins, 3^{rd} edition, 1995). Pharmacokinetic analysis may be performed by noncompartmental (model independent) or compartmental methods. The amount of a compound in a biological fluid (blood, plasma urine) may be measured using well-known methods including such as mass spectrometry. In an embodiment, a GRF molecule and a CYP-metabolized compound are suitable for combination therapy if the maximal observed analyte concentration in plasma (Cₘₐₓ) and the area under the concentration-time curve (AUC) of either compound does not decrease by more than 20% or does not increase by more than 20% in the presence versus in the absence of the other compound of the pair. In another embodiment, a GRF molecule and a GYP-metabolized compound are suitable for combination therapy if the ratio of (A) the AUC₀₋ₜ, AUC_{0-inf} and/or Cₘₐₓ values in presence of the other compound and (B) the AUC₀₋ₜ, AUC_{0-inf} and/or Cₘₐₓ values in the absence of the other compound is between about 0.85 to about 1.2 (i.e., about 85% to about 120%), for example between about 0.90 to about 1.1 (i.e. about 90% to about 110%). In another embodiment, a GRF molecule and a CYP-metabolized compound are suitable for combination therapy if the 90% confidence intervals (CIs) for the ratios of (A) the AUC₀₋ₜ, AUC_{0-inf} and/or Cₘₐₓ values in presence of the other compound and (B) the AUC₀₋ₜ, AUC_{0-inf} and/or Cₘₐₓ values in the absence of the other compound is between about 0.75 to about 1.25 (i.e. about 75% to about 125%), for example between about 0.80 to about 1.25 (i.e., about 80% to about 125%), for example between about 0.90 to about 1.1 (i.e. about 90 to about 110%).

The present inventors have determined that a GRF molecule is suitable for combined therapy with a CYP-metabolized compound, i.e., with no or substantially no drug interaction. Therefore, in an embodiment, the invention further provides a method comprising providing information (e.g., to a subject or a caregiver of the subject) that a GRF molecule and a CYP-metabolized compound may be co-administered to the subject. In an embodiment, the method further comprises informing the subject or the caregiver of the subject that administration of the GRF molecule will have no or substantially no effect on the pharmacokinetics of the CYP-metabolized compound, or that no modification of the treatment regimen (e.g., drug combination, dosage and/or frequency of administration) of the GYP-metabolized compound is necessary.

The invention further provides a use of a GRF molecule for the above-noted combination therapy or to prevent or treat the above-noted conditions. The invention further provides a use of a GRF molecule for the preparation of a medicament for the above-noted combination therapy or to prevent or treat the above-noted conditions. The invention further provides a GRF molecule for use in the above-noted combination therapy or to prevent or treat the above-noted conditions.

In an embodiment the GRF molecule is (hexenoyl trans-3)hGRF(1-44)NH₂. In an embodiment, the above-mentioned GRF molecule (e.g., (hexenoyl trans-3)hGRF(1-44)NH₂) is administered at a daily dose of about 1 mg to about 2 mg, in a further embodiment at a daily dose of about 2 mg. In an embodiment, the GRF molecule (e.g., (hexenoyl trans-3)hGRF(1-44)NH₂) is administered subcutaneously.

In an embodiment, the CYP-metabolized compound is a statin, in a further embodiment, simvastatin (CAS number: 79902-63-9; IUPAC name: (1S,3R,7S,8S,8aR)-8-{2-[(2R,4R)-4-hydroxy-6-oxooxan-2-yl]ethyl}-3,7-dimethyl-1,2,3,7,8,8a-hexahydronaphthalen-1-yl 2,2-dimethylbutanoate).

In an embodiment, the above-mentioned method, use, (hexenoyl trans-3)hGRF(1-44)NH₂ or composition further comprises, prior to said treating (with a GRF molecule such as (hexenoyl trans-3)hGRF(1-44)NH₂), identifying or selecting a subject who is undergoing or who is a candidate for a treatment regimen with a CYP-metabolized compound. The present inventors have determined that no or substantially no drug interaction following administration of a GRF molecule (hexenoyl trans-3)hGRF(1-44)NH₂) with a CYP-metabolized compound, and thus that subjects in need of a treatment to increase GH levels (e.g., to treat conditions in which increasing GH levels is beneficial) but who are undergoing a CYP-metabolized compound-based therapy (or who are a candidate for undergoing such therapy) may be identified or selected for treatment with a GRF molecule such as (hexenoyl trans-3)hGRF(1-44)NH₂.

As such, in an embodiment, there is also provided a method comprising:
identifying or selecting a subject who is (i) undergoing undergoing or who is a candidate for a treatment regimen with a CYP-metabolized compound (e.g., a statin such as simvastatin), and (ii) in need of a treatment to increase GH levels (e.g., to treat conditions in which increasing GH levels is beneficial [e.g., a condition associated with fat accumulation or hypercholesterolemia, such as excess abdominal fat in an HIV-infected subject]);
administering to said subject a GRF molecule such as (hexenoyl trans-3)hGRF(1-44)NH₂,
   wherein said method does not significantly affect pharmacokinetics or clearance of said CYP-metabolized compound, and/or wherein said method does not require modifying said CYP-metabolized compound treatment regimen.

The terms “subject” and “patient” are used interchangeably herein, and include a subject in need of the treatment described herein. In an embodiment, the subject is a mammal, in a further embodiment, a human.

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. In the claims, the word “comprising” is used as an open-ended term, substantially equivalent to the phrase “including, but not limited to”. The articles “a” and “an” are used herein to refer to one or to more than one (i.e., to at least one) of the grammatical object of the article. The term “such as” is used herein to mean, and is used interchangeably, with the phrase “such as but not limited to”. Throughout this application, various references are referred to describe more fully the state of the art to which this invention pertains. The disclosures of these references are hereby incorporated by reference into the present disclosure.

The following examples are illustrative of various aspects of the invention, and do not limit the broad aspects of the invention as disclosed herein.

### EXAMPLES

### Example 1

### Materials and Methods

### Study Drugs:

(hexenoyl trans-3)hGRF(1-44)NH₂: The GRF analog used in the studies described herein is (hexenoyl trans-3)hGRF(1-44)NH₂ (also referred to as [trans-3-hexenoyl]hGRF (1-44) amide and TH9507 herein), which is a synthetic human growth hormone releasing factor analog that comprises the 44-amino acid sequence of human growth hormone releasing factor (hGRF) on which a hexenoyl moiety, a C₆ side chain has been anchored on Tyr 1 at the N-terminal. (hexenoyl trans-3)hGRF(1-44)NH₂ or TH9507 has the following structure:

(SEQ ID NO: 7)

(trans)CH₃—CH₂—CH═CH—CH₂—CO-Tyr-Ala-Asp-Ala-Ile-
Phe-Thr-Asn-Ser-Tyr-Arg-Lys-Val-Leu-Gly-Gln-Leu-
Ser-Ala-Arg-Lys-Leu-Leu-Gln-Asp-Ile-Met-Ser-Arg-
Gln-Gln-Gly-Glu-Ser-Asn-Gln-Glu-Arg-Gly-Ala-Arg-
Ala-Arg-Leu-NH₂.

(hexenoyl trans-3)hGRF(1-44)NH₂ is synthesized using FMOC solid phase peptide synthesis starting with Ramage Tricyclic Amide Resin. Protected amino acids and trans-3-hexenoyl acid are used for coupling whereby each protected amino acid and trans-3-hexenoyl acid is dissolved in DMF-treated with aluminum oxide with TBTU to assist in reducing racemization and DIPEA to promote activation before coupling. Completeness of couplings is monitored by the Kaiser ninhydrin test (E. Kaiser et al., Anal. Biochem. “Color Test for Detection of Free Terminal Amino Groups in the Solid Phase Synthesis of Peptides”) and the TNBS test (Means and Feeney, 1971, Holden-Day Inc. San Francisco “Chemical Modification of Proteins” p. 217).

The side chain protecting groups and the peptide-resin bond are cleaved by stirring the protected peptide-resin in a cleavage cocktail consisting of 90% TFA, 5% EDT and 5% water. The crude peptide is purified by HPLC through a three-stage purification scheme using the following buffers, 0.1% MSA, TEAP pH 6.5 and 2% HOAc affording pure [trans-3-hexenoyl]hGRF (1-44) amide (≧98.5%). The purified peptide lots are pooled and reconstituted in 0.5% acetic acid and lyophilized.

Lyophilization Process. The samples were lyophilized by freezing at −50° C. and holding, annealing to −10° C. and holding, primary drying at −10° C. under 100 mTorr and secondary drying at 25° C. under 100 mTorr.

2 ml of TH9507 (1 mg/ml injectable solution) was administered by subcutaneous injection under fasting conditions once daily for 7 consecutive days (daily dose of 2 mg TH9507).

Simvastatin: One Zocor® 80 mg tablet (simvastatin) by Merck Frosst Canada Ltd., under fasting conditions, as indicated below.

### Subjects:

N=58 healthy adult (male and female) subjects were enrolled. Dosing occurred in two groups.

### Example 2

### Methods and Results

In two randomized, open-label, two-way crossover studies, subjects were administered 2 mg of TH9507 on days 1 to 7, with 80 mg simvastatin (N=58) co-administered on Day 6 (Treatment A), and a single dose of simvastatin alone on day 6 (Treatment B) in a crossover manner. PK samples were collected on day 6, and simvastatin and TH9507 plasma concentrations were measured. The A/B ratios and 90% confidence intervals (CI) within 80-125% would be indicative that TH9507 has no clinically significant impact on simvastatin PKs. Administration of drugs and collection of samples was performed as indicated in Table I. Treatment A relates to administration of TH9507 and simvastatin and Treatment B relates to administration of simvastatin alone.

TABLE I
Administration of drugs and collection of samples

| | Treatment A | Treatment B |
|---|---|---|
| Days 1 | Administer TH9507 | — |
| to 5 | | |
| Day 6 | Administer TH9507; collect samples | — |
| | at 0, 0.1, 0.15, 0.2, 0.25, and 0.5 h | |

Administer simvastatin; collect samples at 0, 0.5,
1, 1.25, 1.5, 1.75, 2, 2.25, 2.5, 3, 3.5, 4, 4.5, 5,
5.5, 6, 7, 8, 10, 12, 16, 24, 30, 36, and 48 h

| | | |
|---|---|---|
| Day 7 | Administer TH9507 | — |

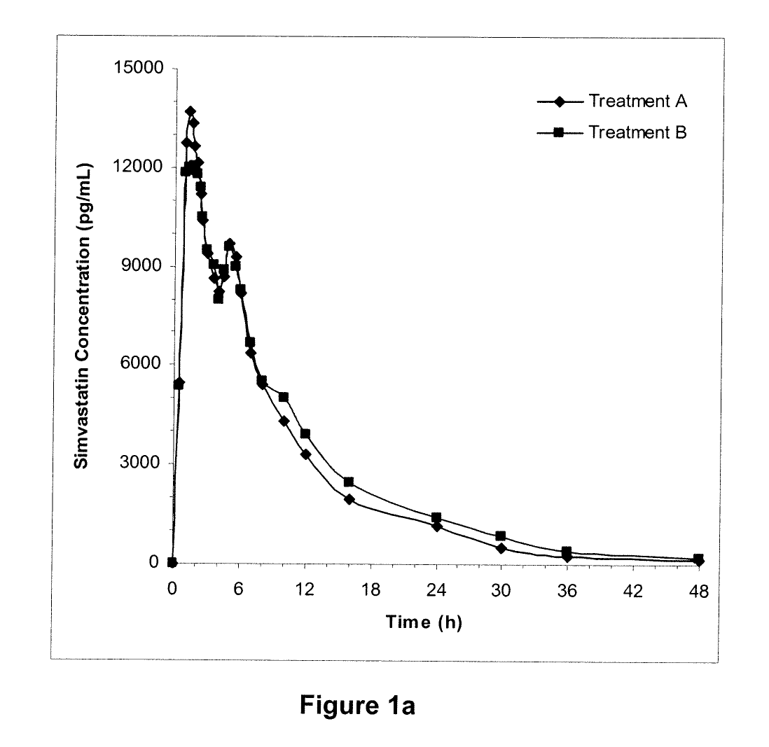

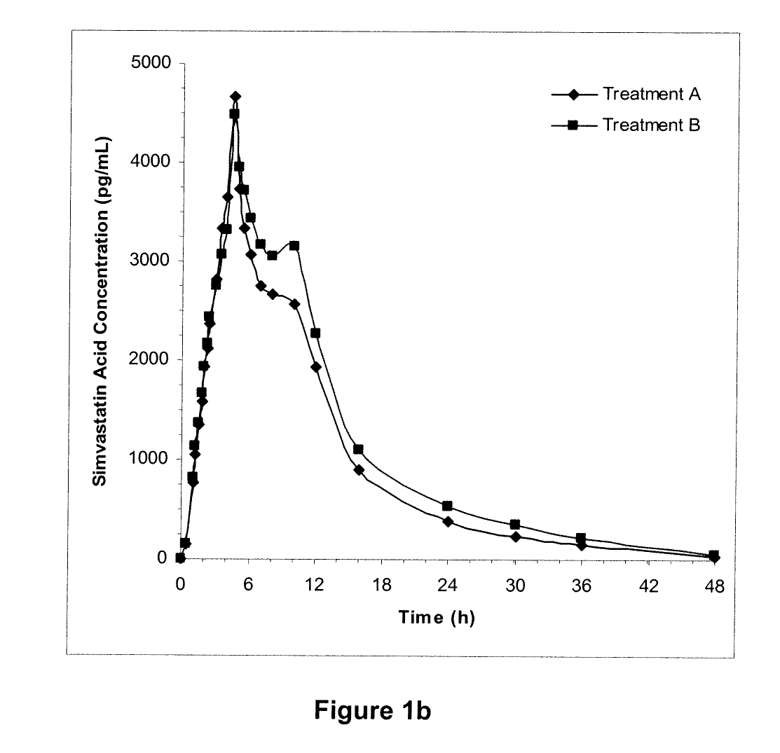

Bioanalytical: ELISA was used to measure plasma TH9507 concentrations and LC/MS/MS was used to measure plasma concentrations of simvastatin and simvastatin acid. Concentration profiles of plasma simvastatin and simvastatin acid are shown in FIGS. 1a and 1b, respectively.

PK/statistics: PK parameters were calculated using standard noncompartmental approaches.

ANOVAs on natural log-transformed AUC₀₋ₜ, AUC_{0-inf} and Cₘₐₓ were conducted for simvastatin, simvastatin acid. Although a significant treatment-by-group interaction was found for AUC₀₋ₜ and AUC_{0-inf} for both simvastatin and simvastatin acid, no clinically-based reason for the group difference was evident, and data from both groups were pooled together. The final ANOVA models included group, sequence, treatment, and period nested within group as fixed effects and subject nested within group-by-sequence as a random effect. The treatment-by-group term was excluded from the models for the groups combined, to be conservative. The 90% CIs for the Treatment A/Treatment B LSM ratios for AUC₀₋ₜ, AUC_{0-inf} and Cₘₐₓ were to be within 80-125% in order to conclude that there is no clinically significant impact of TH9507 on simvastatin PK.

Blood sampling for TH9507 was designed to only estimate the PK profile; descriptive statistics were calculated for TH9507 plasma concentration data to demonstrate that the exposure was in the range of the TH9507 plasma concentration data observed in subjects who did not receive simvastatin.

TABLE 2
Summary of ANOVA results and PK parameters
for simvastatin and simvastatin acid

| | | | | 90% Confi- |
|---|---|---|---|---|
| | Treatment Means | | A/B | dence |
| Parameter | Treatment A | Treatment B | Ratio (%) | Interval (%) |

Simvastatin

| | | | | |
|---|---|---|---|---|
| AUC₀₋ₜ | 94731.59 | 102715.03 | 92.2 | 83.5-101.9 |
| (pg · h/mL)^{a} | | | | |
| AUC_{0-inf} | 97748.35 | 106622.76 | 91.7 | 83.0-101.3 |
| (pg · h/mL)^{a} | | | | |
| Cmax | 16372.51 | 15552.09 | 105.3 | 94.6-117.1 |
| (pg/mL)^{a} | | | | |
| Tₘₐₓ (h)^{b} | 1.50 | 1.50 | — | — |
| | (0.50-5.50) | (0.50-10.00) | | |
| T_{1/2} (h)^{c} | 7.62 (53.5) | 8.56 (64.3) | — | — |

Simvastatin Acid

| | | | | |
|---|---|---|---|---|
| AUC₀₋ₜ | 37466.20 | 43438.29 | 86.3 | 80.2-92.7 |
| (pg · h/mL)^{a} | | | | |
| AUC_{0-inf} | 39906.42 | 46833.23 | 85.2 | 78.6-92.4 |
| (pg · h/mL)^{a} | | | | |
| Cₘₐₓ (pg/mL)^{a} | 4283.93 | 4327.58 | 99.0 | 91.9-106.7 |
| Tₘₐₓ (h)^{b} | 4.50 | 4.50 | — | — |
| | (4.00-12.00) | (2.03-12.03) | | |
| T_{1/2} (h)^{c} | 6.91 (69.5) | 8.07 (53.0) | — | — |

^{a}Geometric mean calculated by exponentiating the LSM from a model using log-transformed response;
^{b}Median (range);
^{c}Arithmetic mean (% CV)

TABLE 3
Summary of PK parameters for TH9507 in simvastatin studies

| Parameter | Mean | |
|---|---|---|
| AUC₀₋ₜ (pg · h/mL) ^{a} | 888.4 | (37.1) |
| AUC_{0-inf} (pg · h/mL) ^{a} | 1052.7 | (37.8) |
| Cₘₐₓ (pg/mL) ^{a} | 3356.3 | (33.8) |
| Tₘₐₓ (h) ^{b} | 0.15 | (0.10-0.20) |
| T_{1/2} (h) ^{c} | 0.14 | (42.0) |

^{a} Geometric mean (% CV);
^{b} Median (range);
^{c} Arithmetic mean (% CV)

For simvastatin, the acceptance limits of 80-125% for the ratio and 90% CI of Treatment A/Treatment B were met for AUC₀₋ₜ, AUC_{0-inf} and Cₘₐₓ; ratios of least squares geometric means and corresponding 90% CIs for AUC₀₋ₜ, AUC_{0-inf} and Cₘₐₓ were contained within the acceptance range. For simvastatin acid, only the lower CI for AUC_{0-inf} (78.6%) fell slightly outside the range.

These studies demonstrate that the impact of TH9507 on CYP3A activity is not significant. Therefore, simvastatin may be administered in conjunction with TH9507 without any change in their dosing regimen.

TABLE 4
Abbreviations used herein

| | |
|---|---|
| AE | Adverse Event |
| ANOVA | Analysis of Variance |
| AUC | Area under the concentration-time curve |
| AUC_{0-inf} | Area under the concentration-time curve from time |
| | zero to infinity (extrapolated) |
| AUC₀₋ₜ | Area under the concentration-time curve, from time |
| | zero to time of last non-zero concentration |
| CI | Confidence interval |
| Cₘₐₓ | Maximal observed analyte concentration in plasma |
| CPK | Creatine phosphokinase |
| CV | Coefficient of variation |
| CYP | Cytochrome P450 |
| CYP 3A | Cytochrome P450 3A |
| CYP 3A4 | Cytochrome P450 3A4 (similary, CYP 3A5 or CYP 3A7 |
| | refer to cytochrome P450 3A5 or 3A7, respectively) |
| DIPEA | N,N-diisopropylethylamine |
| DMF | N,N-dimethylformamide |
| EDT | 1,2-ethane dithiol |
| ELISA | Enzyme-linked immunosorbent assay |
| FMOC | 9-fluorenylmethyloxycarbonyl |
| (h)GH | (human) Growth Hormone |
| (h)GRF | (human) Growth Hormone-Releasing Factor |
| GHRH | Growth Hormone-Releasing Hormone |
| HIV | Human Immunodeficiency Virus |
| HPLC | High-performance liquid chromatography |
| LC/MS/MS | Liquid Chromatography/Mass Spectrometry/Mass |
| | Spectrometry |
| MSA | methane sulfonic acid |
| PK | Pharmacokinetic |
| SAE | Severe adverse event |
| SD | Standard Deviation |
| T_{1/2} | The apparent first-order terminal elimination half- |
| | life |
| TBTU | 2-(1H-benzotriazole-1-yl)-1,1,3,3-tetramethyluronium |
| | tetrafluoroborate |
| TEAP | triethylammonium phosphate |
| TFA | trifluoroacetic acid |
| Tₘₐₓ | Time of observed Cₘₐₓ |
| TNBS | trinitrobenzene sulfonic acid |

Although the present invention has been described hereinabove by way of specific embodiments thereof, it can be modified, without departing from the spirit and nature of the subject invention as defined in the appended claims.

1. A method of inducing GH levels in a subject undergoing a treatment regimen with or who is a candidate for a treatment regimen with a CYP-metabolized compound, without modifying said treatment regimen, said method comprising administering to said subject an effective amount of (hexenoyl trans-3)hGRF(1-44)NH₂.

2. A method of treating excess abdominal fat in a HIV-infected subject with lipodystrophy, wherein said subject is undergoing treatment with or is a candidate for treatment with a CYP-metabolized compound, without modifying said treatment regimen, said method comprising administering to said subject an effective amount of (hexenoyl trans-3)hGRF(1-44)NH₂.

3. The method of claim 1, wherein said method further comprises selecting a subject who is undergoing, or who is a candidate for, a treatment regimen with a CYP-metabolized compound.

4. The method of claim 2, wherein said method further comprises selecting a subject who is undergoing, or who is a candidate for, a treatment regimen with a CYP-metabolized compound.

5. A method comprising providing information to a subject or to a caregiver of the subject that (hexenoyl trans-3)hGRF(1-44)NH₂ and a CYP-metabolized compound can be co-administered to the subject.

6. The method of claim 5, further comprising providing information to the subject or to the caregiver of the subject that (hexenoyl trans-3)hGRF(1-44)NH₂ can be co-administered to the subject without significantly affecting pharmacokinetics of the CYP-metabolized compound.

7. The method of claim 5, further comprising providing information to the subject or to the caregiver of the subject that (hexenoyl trans-3)hGRF(1-44)NH₂ can be co-administered to the subject without modifying the treatment regimen of the CYP-metabolized compound.

8. The method of any one of claims 5, further comprising selecting a subject who is undergoing, or who is a candidate for, a treatment regimen with (hexenoyl trans-3)hGRF(1-44)NH₂ and with a CYP-metabolized compound.

9. The method of claim 1, wherein the CYP-metabolized compound is a statin.

10. The method of claim 9, wherein the statin is simvastatin.

11. The method of claim 2, wherein the GYP-metabolized compound is a statin.

12. The method of claim 11, wherein the statin is simvastatin.

13. The method of claim 5, wherein the CYP-metabolized compound is a statin.

14. The method of claim 13, wherein the statin is simvastatin.

15. The method of claim 1, wherein the (hexenoyl trans-3)hGRF(1-44)NH₂ is administered at a daily dose of about 2 mg.

16. The method of claim 1, wherein the (hexenoyl trans-3)hGRF(1-44)NH₂ is administered subcutaneously.

17. The method of claim 2, wherein the (hexenoyl trans-3)hGRF(1-44)NH₂ is administered at a daily dose of about 2 mg.

18. The method of claim 2, wherein the (hexenoyl trans-3)hGRF(1-44)NH₂ is administered subcutaneously.

19. The method of claim 5, wherein the (hexenoyl trans-3)hGRF(1-44)NH₂ is administered at a daily dose of about 2 mg.

20. The method of claim 5, wherein the (hexenoyl trans-3)hGRF(1-44)NH₂ is administered subcutaneously.
